(19) United States
(12) Patent Application Publication
(10) Pub. No.: US 2023/0364144 A1
(43) Pub. Date: Nov. 16, 2023

(54) STIMULATION OF OVARIAN FUNCTION SUBSEQUENT TO CHEMOTHERAPY AND/OR RADIATION THERAPY USING NATURAL KILLER CELLS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/311,474

(22) Filed: May 3, 2023

Related U.S. Application Data
(60) Provisional application No. 63/340,447, filed on May 10, 2022.

(51) Int. Cl. A61K 35/17; C12N 5/0783

(52) U.S. Cl. CPC A61K 35/17; C12N 5/0646; C12N 2501/065; C12N 2501/2312; C12N 2501/2315; C12N 2506/45; C12N 2500/02

(57) ABSTRACT

Described are protocols, compositions of matter and therapeutic means useful for treatment of ovarian failure caused by oncology treatments. The utilization of natural killer cells allows for dual activity of tissue regeneration in the ovary, while concurrently inhibiting possibility of oncology relapse. In embodiments NK cells are injected as an adjuvant to stem cell and/or regenerative cell therapy to not only enhance therapeutic effect but also to reduce probability of tumor relapse.

### CROSS-REFERENCE TO RELATED APPLICATIONS

This application claims priority to U.S. Provisional Application No. 63/340,447, titled “Stimulation of Ovarian Function Subsequent to Chemotherapy and/or Radiation Therapy using Natural Killer Cells” filed May 10, 2022, which is hereby incorporated by reference herein in its entirety.

### FIELD OF THE INVENTION

The invention is directed to methods of treating and preventing ovarian failure by administering Natural Killers cells to a patient in need after chemotherapy and/or radiation therapy.

### BACKGROUND OF THE INVENTION

Natural killer (NK) cells are typically seen as inflammatory and cancer killing. Numerous studies have shown correlation between tumor infiltration by NK cells and increase patient survival [1-13]. The original studies of NK cells in the 1980s identified them as lymphocytes that phenotypically were neither T cells nor B cells and were able to spontaneously, without prior sensitization, eliminate virally infected or malignant cells in vitro and in vivo [14-18]. Markers associated with NK cells include CD56 and CD16. It is known that NK cells possess the ability to recognize alterations in the “immunological self” through a diverse set of germline-encoded activating and inhibitory receptors and display a broad range of effector functions that play important roles in responding to infections, malignancies and allogeneic tissue. NK recognition involves activation in response to sensing of cellular stressors in cells, which occurs in numerous cancers, as well as reduction in HLA molecules, which is a product of viral infections, or in cases of allogeneic tissue, NK cells are activated by not recognizing self-HLA. Unfortunately, the use of NK cells has not used for the purpose of ovarian repair/regeneration.

### SUMMARY

Preferred methods are directed to methods of treating a ovarian degeneration comprising administration of natural killer (NK) cells which have been modified to allow for enhanced regenerative activity wherein said modification comprising contacting said NK cells with a regenerative cell population.

Preferred embodiments are directed to methods wherein said natural killer cell are either autologous, allogeneic, or xenogeneic to said patient.

Preferred embodiments are directed to methods wherein said natural killer cells comprise a natural killer cell line.

Preferred embodiments are directed to methods wherein said natural killer cell line is NK-92 or a derivative thereof.

Preferred embodiments are directed to methods wherein said natural killer cell is derived from an expanded natural killer cell progenitor.

Preferred embodiments are directed to methods wherein said natural killer cell is derived from a pluripotent stem cell.

Preferred embodiments are directed to methods wherein said pluripotent stem cell is an inducible pluripotent stem cell.

Preferred embodiments are directed to methods wherein said pluripotent stem cell is an embryonic stem cell.

Preferred embodiments are directed to methods wherein said pluripotent stem cell is a somatic cell nuclear transfer derived stem cell.

Preferred embodiments are directed to methods wherein said pluripotent stem cell is a parthenogenically derived stem cell.

Preferred embodiments are directed to methods wherein said enhanced survival and/or activity is accomplished through treatment of said natural killer cells with hypoxic preconditioning.

Preferred embodiments are directed to methods wherein said hypoxic preconditioning comprises exposure of said natural killer cells to conditions allowing for upregulation of HIF-1 alpha gene expression by 25% or more as compared to baseline culture.

Preferred embodiments are directed to methods wherein said hypoxic preconditioning comprises exposure of said natural killer cells to conditions allowing for upregulation of HIF-1 alpha gene expression by 50% or more as compared to baseline culture.

Preferred embodiments are directed to methods wherein said hypoxic preconditioning comprises exposure of said natural killer cells to conditions allowing for nuclear translocation of HIF-1 alpha protein by 25% or more as compared to baseline culture.

Preferred embodiments are directed to methods wherein said hypoxic preconditioning comprises exposure of said natural killer cells to conditions allowing for nuclear translocation of HIF-1 alpha protein by 50% or more as compared to baseline culture.

Preferred embodiments are directed to methods wherein hypoxic preconditioning involves culturing of said cells with agents capable of imitating hypoxia.

Preferred embodiments are directed to methods wherein said hypoxic preconditioning involves culture of cells with carbon monoxide.

Preferred embodiments are directed to methods wherein said culture with carbon monoxide is exposing cells to a gas composition comprising: a. 0% to about 79% by weight nitrogen gas; b. about 21% to about 100% by weight oxygen gas; and c. about 0.0000001% to less than 0.3% by weight carbon monoxide gas.

Preferred embodiments are directed to methods wherein said composition contains 0% nitrogen and about 100% oxygen.

Preferred embodiments are directed to methods wherein said composition contains 0% nitrogen and the amount of carbon monoxide in said composition ranges from about 0.0001% to about 0.075%.

Preferred embodiments are directed to methods wherein the amount of carbon monoxide in said composition ranges from about 0.0001% to about 0.075%.

Preferred embodiments are directed to methods wherein said composition contains 0% nitrogen and the amount of carbon monoxide in said composition ranges from about 0.005% to about 0.05%.

Preferred embodiments are directed to methods wherein the amount of carbon monoxide in said composition ranges from about 0.005% to about 0.05%.

Preferred embodiments are directed to methods wherein said hypoxic preconditioning is performed by culturing natural killer cells or progeny thereof in conditions of 1% to 15% of oxygen in terms of volume ratio.

Preferred embodiments are directed to methods wherein said cells are cultured for at least one passage.

Preferred embodiments are directed to methods wherein modification of said NK cell to allow for enhanced survival and/or activity constitutes culturing said NK cell with an epigenetic modulator.

Preferred embodiments are directed to methods wherein said epigenetic modulator comprises a histone deacetylase inhibitor.

Preferred embodiments are directed to methods wherein said histone deacetylase inhibitor is valproic acid.

Preferred embodiments are directed to methods wherein said histone deacetylase inhibitor is vorinostat.

Preferred embodiments are directed to methods wherein said histone deacetylase inhibitor is entinostat.

Preferred embodiments are directed to methods wherein said histone deacetylase inhibitor is panobinostat.

Preferred embodiments are directed to methods wherein said histone deacetylase inhibitor is trichostatin A.

Preferred embodiments are directed to methods wherein said histone deacetylase inhibitor is mocetinostat.

Preferred embodiments are directed to methods wherein said histone deacetylase inhibitor is belinostat.

Preferred embodiments are directed to methods wherein said histone deacetylase inhibitor is FK228.

Preferred embodiments are directed to methods wherein said histone deacetylase inhibitor is MC1568.

Preferred embodiments are directed to methods wherein said histone deacetylase inhibitor is tubastatin.

Preferred embodiments are directed to methods wherein said histone deacetylase inhibitor is sodium butyrate.

Preferred embodiments are directed to methods wherein said histone deacetylase inhibitor is sulforaphane.

Preferred embodiments are directed to a natural killer cell modified in a manner to increase resistance to hypoxic environments.

Preferred embodiments are directed to methods and compositions wherein said natural killer cell are either autologous, allogeneic, or xenogeneic to said patient.

Preferred embodiments are directed to methods and compositions wherein said natural killer cells comprise a natural killer cell line.

Preferred embodiments are directed to methods and compositions wherein said natural killer cell line is NK-92 or a derivative thereof.

Preferred embodiments are directed to methods and compositions wherein said natural killer cell is derived from an expanded natural killer cell progenitor.

Preferred embodiments are directed to methods and compositions wherein said natural killer cell is derived from a pluripotent stem cell.

Preferred embodiments are directed to methods and compositions wherein said pluripotent stem cell is an inducible pluripotent stem cell.

Preferred embodiments are directed to methods and compositions wherein said pluripotent stem cell is an embryonic stem cell.

Preferred embodiments are directed to methods and compositions wherein said pluripotent stem cell is a somatic cell nuclear transfer derived stem cell.

Preferred embodiments are directed to methods and compositions wherein said pluripotent stem cell is a parthenogenically derived stem cell.

Preferred embodiments are directed to methods and compositions wherein said enhanced survival and/or activity is accomplished through treatment of said natural killer cells with hypoxic preconditioning.

Preferred embodiments are directed to methods and compositions wherein said hypoxic preconditioning comprises exposure of said natural killer cells to conditions allowing for upregulation of HIF-1 alpha gene expression by 25% or more as compared to baseline culture.

Preferred embodiments are directed to methods and compositions wherein T cells are generated to possess a hypoxia resistant phenotype instead of NK cells.

Preferred embodiments are directed to methods and compositions wherein said NK cells are cultured with regenerative cells.

Preferred embodiments are directed to methods and compositions wherein said culture comprises addition of interleukin-2.

Preferred embodiments are directed to methods and compositions wherein said culture comprises addition of interleukin-4.

Preferred embodiments are directed to methods and compositions wherein said culture comprises addition of anti-CD3.

Preferred embodiments are directed to methods and compositions wherein said culture comprises addition of interleukin-7.

Preferred embodiments are directed to methods and compositions wherein said culture comprises addition of interleukin-12.

Preferred embodiments are directed to methods and compositions wherein said culture comprises addition of GM-CSF.

Preferred embodiments are directed to methods and compositions wherein said culture comprises addition of interleukin-15.

Preferred embodiments are directed to methods and compositions wherein said culture comprises addition of interleukin-18.

Preferred embodiments are directed to methods and compositions wherein said culture comprises addition of interleukin-21.

Preferred embodiments are directed to methods and compositions wherein said culture comprises addition of interleukin-23.

Preferred embodiments are directed to methods and compositions wherein said culture comprises addition of culture supernatant from said regenerative cells to said NK cells.

Preferred embodiments are directed to methods and compositions wherein said regenerative cells are activated prior to contact with said NK cells.

Preferred embodiments are directed to methods and compositions wherein said regenerative cells are activated by contact with allogeneic T cells.

Preferred embodiments are directed to methods and compositions wherein said regenerative cells are activated by culture with an inflammatory cytokine.

Preferred embodiments are directed to methods and compositions wherein said regenerative cells are activated by culture with an activator of NF-kappa B.

Preferred embodiments are directed to methods and compositions wherein said regenerative cells are activated by culture with an activator of the JAK-STAT pathway.

Preferred embodiments are directed to methods and compositions wherein said regenerative cells are activated by culture with an activator of NF-kappa B.

Preferred embodiments are directed to methods and compositions wherein said regenerative cells are activated by culture with an activator of the toll like receptor pathway.

Preferred embodiments are directed to methods and compositions wherein said regenerative cells are activated by culture with an activator of the retinoic acid inducible gene.

Preferred embodiments are directed to methods and compositions wherein said regenerative cells are activated by culture with an activator of the melanoma differentiation associated gene-5.

Preferred embodiments are directed to methods and compositions wherein said regenerative cells are activated by culture with an activator of the nucleotide-binding oligomerization domain-containing protein.

Preferred embodiments are directed to methods and compositions wherein said regenerative cells are activated by treatment with interleukin-1.

Preferred embodiments are directed to methods and compositions wherein said regenerative cells are activated by treatment with interferon alpha.

Preferred embodiments are directed to methods and compositions wherein said regenerative cells are activated by treatment with interferon beta.

Preferred embodiments are directed to methods and compositions wherein said regenerative cells are activated by treatment with interferon gamma.

Preferred embodiments are directed to methods and compositions wherein said regenerative cells are activated by treatment with interferon omega.

Preferred embodiments are directed to methods and compositions wherein said regenerative cells are activated by treatment with Poly IC.

Preferred embodiments are directed to methods and compositions wherein said regenerative cells are activated by treatment with lipopolysaccharide.

Preferred embodiments are directed to methods and compositions wherein said regenerative cells are activated by treatment with low molecular weight hyaluronic acid.

Preferred embodiments are directed to methods and compositions wherein said regenerative cells are activated by treatment with CpG motifs.

Preferred embodiments are directed to methods and compositions wherein said regenerative cells are activated by treatment with neutrophil extracellular traps.

Preferred embodiments are directed to methods and compositions wherein said regenerative cell is a stem cell.

Preferred embodiments are directed to methods and compositions wherein said stem cell is a hematopoietic stem cell.

Preferred embodiments are directed to methods and compositions wherein said hematopoietic stem cell is capable of generating leukocytic, lymphocytic, thrombocytic and erythrocytic cells when transplanted into an immunodeficient animal.

Preferred embodiments are directed to methods and compositions wherein said hematopoietic stem cell is non-adherent to plastic.

Preferred embodiments are directed to methods and compositions wherein said hematopoietic stem cell is adherent to plastic.

Preferred embodiments are directed to methods and compositions wherein said hematopoietic stem cell is exposed to hyperthermia.

Preferred embodiments are directed to methods and compositions wherein said hematopoietic stem cell expresses interleukin-3 receptor.

Preferred embodiments are directed to methods and compositions wherein said hematopoietic stem cell expresses interleukin-1 receptor.

Preferred embodiments are directed to methods and compositions wherein said hematopoietic stem cell expresses c-met.

Preferred embodiments are directed to methods and compositions wherein said hematopoietic stem cell expresses mpl.

Preferred embodiments are directed to methods and compositions wherein said hematopoietic stem cell expresses interleukin-11 receptor.

Preferred embodiments are directed to methods and compositions wherein said hematopoietic stem cell expresses G-CSF receptor.

Preferred embodiments are directed to methods and compositions wherein said hematopoietic stem cell expresses GM-CSF receptor.

Preferred embodiments are directed to methods and compositions wherein said hematopoietic stem cell expresses M-CSF receptor.

Preferred embodiments are directed to methods and compositions wherein said hematopoietic stem cell expresses VEGF-receptor.

Preferred embodiments are directed to methods and compositions wherein said hematopoietic stem cell expresses c-kit.

Preferred embodiments are directed to methods and compositions wherein said hematopoietic stem cell expresses CD33.

Preferred embodiments are directed to methods and compositions wherein said hematopoietic stem cell expresses CD133.

Preferred embodiments are directed to methods and compositions wherein said hematopoietic stem cell expresses CD34.

Preferred embodiments are directed to methods and compositions wherein said hematopoietic stem cell expresses Fas ligand.

Preferred embodiments are directed to methods and compositions wherein said hematopoietic stem cell does not express lineage markers.

Preferred embodiments are directed to methods and compositions wherein said hematopoietic stem cell does not express CD14.

Preferred embodiments are directed to methods and compositions wherein said hematopoietic stem cell does not express CD16.

Preferred embodiments are directed to methods and compositions wherein said hematopoietic stem cell does not express CD3.

Preferred embodiments are directed to methods and compositions wherein said hematopoietic stem cell does not express CD56.

Preferred embodiments are directed to methods and compositions wherein said hematopoietic stem cell does not express CD38.

Preferred embodiments are directed to methods and compositions wherein said hematopoietic stem cell does not express CD30.

Preferred embodiments are directed to methods and compositions wherein said regenerative cell is a mesenchymal stem cell.

Preferred embodiments are directed to methods and compositions wherein said mesenchymal stem cells are naturally occurring mesenchymal stem cells.

Preferred embodiments are directed to methods and compositions wherein said mesenchymal stem cells are generated in vitro.

Preferred embodiments are directed to methods and compositions wherein said naturally occurring mesenchymal stem cells are tissue derived.

Preferred embodiments are directed to methods and compositions wherein said naturally occurring mesenchymal stem cells are derived from a bodily fluid.

Preferred embodiments are directed to methods and compositions wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; w) ovarian tissue and x) subepithelial umbilical cord tissue.

Preferred embodiments are directed to methods and compositions wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, and salivary gland serous cells.

Preferred embodiments are directed to methods of treating ovarian failure comprising administration of NK cells into the ovary.

Preferred embodiments are directed to methods and compositions wherein said natural killer cell are either autologous, allogeneic, or xenogeneic to said patient.

Preferred embodiments are directed to methods and compositions wherein said natural killer cells comprise a natural killer cell line.

Preferred embodiments are directed to methods and compositions wherein said natural killer cell line is NK-92 or a derivative thereof.

Preferred embodiments are directed to methods and compositions wherein said natural killer cell is derived from an expanded natural killer cell progenitor.

Preferred embodiments are directed to methods and compositions wherein said natural killer cell is derived from a pluripotent stem cell.

Preferred embodiments are directed to methods and compositions wherein said pluripotent stem cell is an inducible pluripotent stem cell.

Preferred embodiments are directed to methods and compositions wherein said pluripotent stem cell is an embryonic stem cell.

Preferred embodiments are directed to methods and compositions wherein said pluripotent stem cell is a somatic cell nuclear transfer derived stem cell.

Preferred embodiments are directed to methods and compositions wherein said pluripotent stem cell is a parthenogenically derived stem cell.

Preferred embodiments are directed to methods and compositions wherein said enhanced survival and/or activity is accomplished through treatment of said natural killer cells with hypoxic preconditioning.

Preferred embodiments are directed to methods and compositions wherein said hypoxic preconditioning comprises exposure of said natural killer cells to conditions allowing for upregulation of HIF-1 alpha gene expression by 25% or more as compared to baseline culture.

Preferred embodiments are directed to methods and compositions wherein said hypoxic preconditioning comprises exposure of said natural killer cells to conditions allowing for upregulation of HIF-1 alpha gene expression by 50% or more as compared to baseline culture.

Preferred embodiments are directed to methods and compositions wherein said hypoxic preconditioning comprises exposure of said natural killer cells to conditions allowing for nuclear translocation of HIF-1 alpha protein by 25% or more as compared to baseline culture.

Preferred embodiments are directed to methods and compositions wherein said hypoxic preconditioning comprises exposure of said natural killer cells to conditions allowing for nuclear translocation of HIF-1 alpha protein by 50% or more as compared to baseline culture.

Preferred embodiments are directed to methods and compositions wherein hypoxic preconditioning involves culturing of said cells with agents capable of imitating hypoxia.

Preferred embodiments are directed to methods and compositions wherein said hypoxic preconditioning involves culture of cells with carbon monoxide.

Preferred embodiments are directed to methods and compositions wherein said culture with carbon monoxide is exposing cells to a gas composition comprising: a. 0% to about 79% by weight nitrogen gas; b. about 21% to about 100% by weight oxygen gas; and c. about 0.0000001% to less than 0.3% by weight carbon monoxide gas.

Preferred embodiments are directed to methods and compositions wherein said composition contains 0% nitrogen and about 100% oxygen.

Preferred embodiments are directed to methods and compositions wherein said composition contains 0% nitrogen and the amount of carbon monoxide in said composition ranges from about 0.0001% to about 0.075%.

### DETAILED DESCRIPTION OF THE INVENTION

The invention provides natural killer cells that have been generated to possess properties beneficial for ovarian regeneration and/or modifying of inflammation. In one embodiment the invention provides generation of natural killer cells from pluripotent stem cells, and contacting progenitors of natural killer cells with regenerative cells capable of endowing regenerative properties to said natural killer cells.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

“About” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

“Administered” or “administering”, as used herein, refers to any method of providing a composition to an individual such that the composition has its intended effect on the individual. For example, one method of administering is by a direct mechanism such as local tissue administration (including by injection), oral ingestion, transdermal patch, topical, inhalation, suppository etc.

“Allogeneic” refers to tissues or cells from another body that in a natural setting are immunologically incompatible or capable of being immunologically incompatible, although from one or more individuals of the same species.

“Allotransplantation” refers to the transplantation of organs, tissues, and/or cells from a donor to a recipient, where the donor and recipient are different individuals, but of the same species. Tissue transplanted by such procedures is referred to as an allograft or allotransplant.

Allostimulatory” and “alloreactive” refer to stimulation and reaction of the immune system in response to an allologous antigens, or “alloantigens” or cells expressing a dissimilar HLA haplotype.

“Autologous” refers to tissues or cells that are derived or transferred from the same individual's body.

“Autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

“Biologically active” refers to any molecule having structural, regulatory or biochemical functions.

“Cell culture” is an artificial in vitro system containing viable cells, whether quiescent, senescent or (actively) dividing. In a cell culture, cells are grown and maintained at an appropriate temperature, typically a temperature of 37.degree. C. and under an atmosphere typically containing oxygen and CO.sub.2, although in other cases these are altered. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

“Comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

“Drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

“Individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

“One embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject. Methods of the disclosure include the reduction in intensity of disc degeneration and may include the reduction in intensity of one or more symptoms related thereto, including further degeneration, pain, and so forth.

“Therapeutic agent” means to have “therapeutic efficacy” in modulating anticancer activity an amount of the therapeutic is said to be a “immune modulatory amount”, if administration of that amount of the therapeutic is sufficient to cause a significant modulation (i.e., increase or decrease) in anticancer activity when administered to a subject (e.g., an animal model or human patient) needing modulation of immune activity.

“Therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

For practice of the invention disclosed NK cells are generated from in vivo progenitors or in vitro sources. In one embodiment NK cells are generated from pluripotent stem cells. Generation of NK cells from this source is widely known in the literature and is described in the following references: a) cord blood [19-24]; b) bone marrow [25-28]; c) embryonic stem cells [29-31]; d) inducible pluripotent stem cells [32]; and e) peripheral blood [33-36].

In some embodiments NK cells are administered without manipulation into the ovary. In other embodiments, NK cells are activated by hypoxic conditions before administration. Said NK cells can be naturally occurring, or NK cells that have been expanded ex vivo, or NK cell lines such as NK92, or NK cells generated from progenitor cells. In particular embodiments, the hypoxic condition comprises oxygen levels from 0.1%-10%, 0.1%-5%, 0.1%-2.5%, 0.1%-2%, 0.1%-1%, 0.5%-10%, 0.5%-7.5%, 0.5%-5%, 0.5%-2.5%, 0.5%-2%, 0.5%-1%, 1%-10%, 1%-7.5%, 1%-5%, 1%-2.5%, 1%-2%, 2%-10%, 2%-7.5%, 2%-5%, 2%-2.5%, 5%-10%, 5%-7.5%, 5%-6%, or 7.5%-10% oxygen, as examples only. The duration of exposure, including with (but not limited to) these representative levels of oxygen may be for a duration of 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hour (hr), 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3 days, 12 hrs-2 days, 12 hrs-1 day, 12-18 hrs, 12-14 hrs, 1-3 days, or 1-2 days, as examples only.

Said “activation” of NK cells by hypoxia is associated with induction of cell protective enzymes such as heme-oxygenase, superoxide dismutases and NFR-2. Treatment of cells under hypoxic conditions may be accomplished by subjecting cells to low oxygen conditions. In some embodiments of the invention, exposing NK cells to an environmental factor involves exposing the NK cells to reduced oxygen tension. In general terms, this is accomplished by contacting a composition stem cells with an environment that has a low level of ambient oxygen. The phrases “low ambient oxygen conditions,” “low oxygen,” and “reduced oxygen tension” refer to any oxygen concentration that is less than atmospheric oxygen. Low ambient oxygen conditions generally means any oxygen concentration below about 20%, preferably below about 15%, more preferably below about 5-10%, at sea level. Low oxygen conditions may be kept as close as possible to the normal physiological oxygen conditions in which a particular NK cell would be found in vivo. Thus, in some embodiments, the conditions employed for cells will depend on the tissue compartment or regional origin of a particular cell; such conditions are known to the skilled artisan. “Physiologic” oxygen levels are the range of oxygen levels normally found in healthy tissues and organs.

In one embodiment, the low ambient Oxygen conditions comprise an ambient oxygen condition of between about 0.25% to about 18% oxygen. In another embodiment, the ambient oxygen conditions comprise between about 0.5% to about 15% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures.

One aspect of the invention relates to the timing (e.g. stage of cell culture) at which the NK are exposed to low oxygen (i.e. reduced oxygen tension) conditions. One skilled in the art will appreciate that the timing of the exposure of the NK cells to reduced oxygen tension will depend on the NK characteristics that are desired and which result from exposure to the altered environmental condition of low oxygen tension. NK cells may be exposed to reduced oxygen tension at any time during the in vitro culture of the stem cells. NK cells may be exposed to reduced oxygen tension at times including, but not limited to, after collection of the NK cells as a tissue sample, during disaggregation of such tissue sample, during the primary culture of NK cells, during the in vitro expansion of the NK cells (e.g. over multiple cell passages), during priming (e.g. when NK cells are induced to assume a desired biological activity prior to injection into a subject), and combinations thereof.

In some embodiments of the invention, NK cells are exposed to reduced oxygen tension during the in vitro culture of the NK cells. One skilled in the art will appreciate that there are various methods for culturing NK cells under low ambient oxygen conditions (i.e. reduced oxygen tension). For example, suitable processes, reagents and equipment for practicing the invention are disclosed in the following references, which are incorporated herein by reference: U.S. Pat. Nos. 6,759,242; 6,846,641; 6,610,540. Although these references disclose particular procedures and reagents, any low oxygen culture condition capable of expanding stern cells according to the invention may be used.

NK cells can be exposed to low oxygen conditions under any methodology that permits the NK cells to attain an enhanced differentiation potential, proliferation rate, engraftment ability and/or in vivo tumor migratory ability. Specialized laboratory facilities may have completely enclosed environments in which the oxygen levels are controlled throughout a dedicated, isolated room. In such specialized areas, low oxygen levels can be maintained throughout the isolation, growth and differentiation of cells without interruption. Physiologic or low oxygen culturing conditions also can be maintained by using commercially-available chambers which are flushed with a pre-determined gas mixture (e.g., as available from Billups-Rothenberg, San Diego, Calif.). As an adjunct, medium can be flushed with the same gas mixture prior to cell feeding. In general, it is not possible to maintain physiologic or low oxygen conditions during cell feeding and passaging using these smatter enclosed units, and so, the time for these manipulations should be minimized as much as possible. Any sealed unit can be used for physiologic oxygen or low oxygen level culturing provided that adequate humidification, temperature, and carbon dioxide are provided.

In addition to oxygen, the other gases for culture typically are about 5% carbon dioxide and the remainder is nitrogen, but optionally may contain varying amounts of nitric oxide (starting as low as 3 ppm), carbon monoxide and other gases, both inert and biologically active. Carbon dioxide concentrations typically range around 5% as noted above, hut may vary between 2-10%. Both nitric oxide and carbon monoxide are typically administered in very small amounts (i.e. in the ppm range), determined empirically or from the literature.

One aspect of the invention relates to the length of time that the NK cells are exposed to reduced oxygen tension. Under the invention, stem cells may be exposed to reduced oxygen tension for any amount of time that enhances the proliferation and differentiation of the NK cells as disclosed herein. This may be 1 or more hours, 3 or more hours, 6 or more hours, 12 or more hours, or the time may be continuous (e.g. the entire time that the NK cells are cultured in vitro). The temperature during the culture is typically reflective of core body temperature, or about 37 degree. C., but may vary between about 32 degrees centigrade and about 40 degrees centigrade.

In one embodiment, NK cells and/or NK progenitors are exposed to low dose carbon monoxide (CO) as a means of preconditioning prior to administration. CO may also be utilized as a preservative for storing cells prior to administration. It is an unexpected result that the inclusion of low dosage CO in the storage media maintains and/or enhances viability and activity. Thus, in this embodiment of the present invention, an effective amount of CO is bubbled into storage media before cells are placed in the media or shortly thereafter.

In delivering CO concentrations ranging from about 0.001 to about 3,000 ppm pursuant to the present invention, gaseous compositions according to the present invention may be prepared by mixing commercially available compressed air containing CO (generally about 1% CO) with compressed air or gas containing a higher percentage of oxygen (including pure oxygen), and then mixing the gasses in a ratio which will produce a gas containing a desired amount of CO therein. Alternatively, compositions according to the present invention may be purchased pre-prepared from commercial gas companies. In a preferred embodiment, patients are exposed to oxygen (O.sub.2 at varying doses) and CO at a flow rate of about 12 liters/minute in a 3.70 cubic foot glass exposure chamber. To make a gaseous composition containing a pre-determined amount of CO, CO at a concentration of 1% (10,000 ppm) in compressed air is mixed with >98% O.sub.2 in a stainless-steel mixing cylinder, concentrations delivered to the exposure chamber or tubing will be controlled. Because the flow rate is primarily determined by the flow rate of the O.sub.2 gas, only the CO flow is changed to generate the different concentrations delivered to the exposure chamber or tubing. A carbon monoxide analyzer (available from Interscan Corporation, Chatsworth, Calif.) is used to measure CO levels continuously in the chamber or tubing. Gas samples are taken by the analyzer through a portion the top of the exposure chamber of tubing at a rate of 1 liter/minute and analyzed by electrochemical detection with a sensitivity of about 1 ppb to 600 ppm. CO levels in the chamber or tubing are recorded at hourly intervals and there are no changes in chamber CO concentration once the chamber or tubing has equilibrated. CO is then delivered to the patient for a time (including chronically) sufficient to treat the condition and exert the intended pharmacological or biological effect.

In one embodiment natural killer cells are treated under conditions which cause generation of uterine natural killer (uNK) cells. It is known that uNK cells play a fundamental role in creation of the placental vasculature, in part through stimulation of angiogenesis [37-48].

In one embodiment of the invention, NK cells are generated in the presence of regenerative cells such as mesenchymal stem cells. The generation of NK cells can be performed from NK progenitors. The NK progenitors can be generated by recapitulating normal embryonic development of NK cells. NK cells have been shown to share a number of antigenic and functional similarities to T cells that suggest the possibility of common origins. It is known that there are correlations between T cell and NK cell embryonic development in the liver. In one study, freshly isolated fetal NK cells mediated MHC-unrestricted cytotoxicity against NK-sensitive targets and acquired the ability to lyse NK-resistant tumors after overnight culture in interleukin 2. Unlike adult NK cells, freshly isolated fetal liver NK cells and clones derived from these cells, as well as a subset of cord blood NK cells, express substantial levels of CD3 delta and CD3 epsilon proteins in the cytoplasm. Expression of CD3 epsilon and CD delta transcripts and cytoplasmic proteins in fetal NK clones was confirmed by polymerase chain reaction and Western blot analysis. These findings support the concept that NK and T cells may arise from a common progenitor that expresses components of the CD3 complex. Alternatively, it is possible that the cytoplasmic CD3 delta, epsilon+fetal NK cells represent a distinct subpopulation of NK cells that is predominant in the fetus, but replaced by the cytoplasmic CD3 delta, epsilon-adult NK cell population after embryogenesis [49]. Generation of NK cells from fetal liver [50-52], fetal hematopoietic stem cells [53-58], from hematopoietic stem cells [59-63], from iPSC [64, 65], and embryonic stem cells [66-74], have been described and are incorporated by reference.

In one embodiment of the invention, the invention provides a therapeutic composition comprising a population of human hematopoietic stem or progenitor cells which have been differentiated into NL cells possessing regenerative properties by cultured in conditioned media of mesenchymal stem cells, wherein said cells are suspended in a sterile, therapeutically acceptable solution suitable for administration to a patient. The therapeutic composition of the invention comprises a population of human hematopoietic stem or progenitor cell differentiated NK cells wherein the hematopoietic stem or progenitor cells have been contacted ex vivo with one or more agents capable of increasing CXCR4 gene expression in the cells, and where the cells are characterized by a gene expression signature comprising increased expression, relative to non-contacted stem or progenitor cells, of CXCR4. The expression of the CXCR4 gene allows NK cells, within the context of the current invention, to possess enhanced ability to migrate to injured tissue. Augmented expression of the CXCR4 ligand, SDF-1 has been shown in heart attack [75-91], stroke, liver failure [92], and kidney injury [93-96].

The hematopoietic stem or progenitor cells may be characterized based upon increased levels of gene and cell-surface CXCR4 expression. In the therapeutic composition of the invention, gene expression of CXCR4 in the hematopoietic stem or progenitor cells is increased by at least 2, 3, 4, 5, 10, 15, or 20 fold compared to the expression of CXCR4 in non-contacted cells. In one embodiment NK cells are expressing an enhanced concentration of NK cells. In other embodiments NK cells are generated in a manner to produce regenerative properties. In some embodiments said NK cells express HGF after activation of NK receptors.

In some embodiments of the invention NK cells are transfected to produce therapeutic and/or regenerative growth factors. In one embodiment said NK cells are transfected with hCG [97].

The therapeutic composition of the invention, which in one embodiment is regenerative NK cells, may be further characterized by a gene expression signature wherein expression of one or more signature genes selected from the group consisting of hyaluronan synthase 1 (HAS1), GTP-binding protein GEM (GEM), dual specificity protein phosphatase 4 (DUSP4), amphiregulin (AREG), Nuclear receptor related 1 protein (NR4A2), renin (REN), cAMP-responsive element modulator (CREM), collagen, type I, alpha 1 (COL1A1), and Fos-related antigen 2 (FOSL2) is increased, relative to non-contacted cells. In some embodiments of the invention, regenerative NK cells are administered together with low frequency ultrasound in order to enhance regenerative effects through stimulation of SDF-1 production [98].

General principles of maintaining adherent cell cultures are well-known in the art. As appreciated by those skilled in the art, the fibroblast cells may be counted in order to facilitate subsequent plating of the cells at a desired density. Where, as in the present disclosure, the cells after plating may primarily adhere to a substrate surface present in the culture system (e.g., in a culture vessel), the plating density may be expressed as number of cells plated per mm2 or cm2 of the said substrate surface. In practicing the disclosure, after plating of the regenerative cells, the cell suspension is left in contact with the adherent surface to allow for adherence of cells from the cell population to the substrate. In contacting regenerative cells to the adherent substrate, the cells may be advantageously suspended in an environment comprising at least a medium, in the methods of the disclosure typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

The medium may be a suitable culture medium as described elsewhere in this specification. Preferably, the composition of the medium may have the same features, may be the same or substantially the same as the composition of medium used in the ensuing steps of culturing the attached cells. Otherwise, the medium may be different. In some embodiments, the cells from the fibroblast cell population or from tissue explants of the present disclosure, which have adhered to the substrate, preferably in the environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In some embodiments, the fibroblast cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the disclosure may be cultured for no longer than 60 days, or no longer than 50 days, or no longer than 45 days. The tissue explants and regenerative cells may be cultured in the presence of a liquid culture medium. Typically, the medium will comprise a basal medium formulation as known in the art. Many basal media formulations can be used to culture regenerative cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the regenerative cells cultured. In some embodiments, a culture medium formulation may be explants medium (CEM) which is composed of IMDM supplemented with 10% fetal bovine serum (FBS, Lonza), 100 U/ml penicillin G, 100 .mu.g/ml streptomycin and 2 mmol/L L-glutamine (Sigma-Aldrich). Other embodiments may employ further basal media formulations, such as chosen from the ones above.

For use in the fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., beta-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS).

In some embodiments, the regenerative cells of the present disclosure are modified, such as by exposure to a composition, to enhance the ability of the regenerative cells to suppress the production of TGF-beta in Th3 cells. The modification of the regenerative cells may be monitored by assessing the inhibited production of TGF-beta from Th3 cells subsequent to cell to cell contact between modified regenerative cells and Th3 cells. The composition for modifying regenerative cells may comprise oxytocin, in specific cases. In some embodiments, the regenerative cells are exposed to the composition, such as oxytocin, for a period between approximately 1 minute to approximately 4 weeks, or between approximately 2 hours to approximately 1 week, or between approximately 24 hours to approximately 72 hours. The regenerative cells may be exposed to oxytocin at a concentration ranging between approximately 10 nM to approximately 10 μM, or between approximately 100 nM to approximately 1 μM.

As described, the present inventors have identified that by culturing tissue explants and fibroblast cells for time durations as defined above, and in at least some cases using media compositions as described above, a progenitor or stem cell of the disclosure emerges and proliferates. In some embodiments, fibroblast cells of the present disclosure are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing regenerative cells the present disclosure are provided herein. In certain embodiments, the marker proteins used to identify and characterize the stem cells are selected from the list consisting of c-Kit, Nanog, Sox2, Hey1, SMA, Vimentin (including extracellular vimentin), Cyclin D2, Snail, E-cadherin, Nkx2.5, GATA4, CD105, CD90, CD29, CD73, Wt1, CD34, CD45, and a combination thereof.

In one embodiment the invention teaches phenotypically defined MSC which can be isolated from the umbilical cord tissue and defined morphologically and by cell surface markers. By dissecting out the veins and arteries of cord segments and exposing the umbilical cord tissue, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover, the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be affected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of umbilical cord tissue cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1 times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, a-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

In some embodiments of the invention, cells are disclosed that can produce growth factors upon binding with ligands in ovarian tissue. In some embodiments CAR-NK cells are used in which the CAR targets tissue factor (TF). The interaction between the TF-specific CAR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-, CD134L/OX40L/CD252, CD27L/CD70, Fas ligand (FasL), CD3OL/CD153, tumor necrosis factor beta (TNFP)/lymphotoxin-alpha (LTcc), lymphotoxin-beta (LTP), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins, in that they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell is transduced with one co-stimulatory ligand that is 4-1BBL. In certain embodiments, the immunoresponsive cell is transduced with two co-stimulatory ligands that are 4-1BBL and CD80. CARs transduced with at least one co-stimulatory ligand are described in U.S. Pat. No. 8,389,282, which is incorporated by reference in its entirety. Furthermore, a presently disclosed immunoresponsive cell can be further transduced with at least one cytokine, such that the immunoresponsive cell secretes the at least one cytokine as well as expresses the TF-specific CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

### REFERENCES

ADDIN EN.REFLIST 1. Nelson, M. A., et al., Prognostic and therapeutic role of tumor-infiltrating lymphocyte subtypes in breast cancer. Cancer Metastasis Rev, 2021. 40(2): p. 519-536.
2. He, M., et al., The prognostic significance of tumor-infiltrating lymphocytes in cervical cancer. J Gynecol Oncol, 2021. 32(3): p. e32.
3. Bouzidi, L., et al., Prognostic Value of Natural Killer Cells Besides Tumor-Infiltrating

Lymphocytes in Breast Cancer Tissues. Clin Breast Cancer, 2021.
4. Zhang, X., et al., Identification and validation of an immune-related gene signature predictive of overall survival in colon cancer. Aging (Albany NY), 2020. 12(24): p. 26095-26120.
5. Melaiu, 0., et al., Cellular and gene signatures of tumor-infiltrating dendritic cells and natural-killer cells predict prognosis of neuroblastoma. Nat Commun, 2020. 11(1): p. 5992.
6. Zhang, S., et al., Prognostic Significance of Tumor-Infiltrating Natural Killer Cells in Solid Tumors: A Systematic Review and Meta-Analysis. Front Immunol, 2020. 11: p. 1242.
7. Bisheshar, S. K., et al., The prognostic role of NK cells and their ligands in squamous cell carcinoma of the head and neck: a systematic review and meta-analysis. Oncoimmunology, 2020. 9(1): p. 1747345.
8. Wu, Y., et al., Clinical significance of peripheral blood and tumor tissue lymphocyte subsets in cervical cancer patients. BMC Cancer, 2020. 20(1): p. 173.
9. Henriksen, J. R., et al., Favorable prognostic impact of Natural Killer cells and T cells in high-grade serous ovarian carcinoma. Acta Oncol, 2020. 59(6): p. 652-659.
10. Wu, M., et al., Comprehensive characterization of tumor infiltrating natural killer cells and clinical significance in hepatocellular carcinoma based on gene expression profiles. Biomed Pharmacother, 2020. 121: p. 109637.
11. Wang, J., et al., The prognostic landscape of tumor-infiltrating immune cells in cervical cancer. Biomed Pharmacother, 2019. 120: p. 109444.
12. Cursons, J., et al., A Gene Signature Predicting Natural Killer Cell Infiltration and Improved Survival in Melanoma Patients. Cancer Immunol Res, 2019. 7(7): p. 1162-1174.
13. Muntasell, A., et al., NK Cell Infiltrates and HLA Class I Expression in Primary HER2(+) Breast Cancer Predict and Uncouple Pathological Response and Disease-free Survival. Clin Cancer Res, 2019. 25(5): p. 1535-1545.
14. Matera, L., et al., Modulation of in vitro myelopoiesis by LGL: different effects on early and late progenitor cells. J Immunol, 1986. 136(4): p. 1260-5.
15. Funa, K., et al., Human peripheral blood null lymphocytes stimulated by Staphylococcus aureus Cowan I produce atypical acid-labile interferon in vitro. Scand J Immunol, 1985. 21(1): p. 55-62.
16. Nieminen, P. and E. Saksela, A shared antigenic specificity of human large granular lymphocytes and precursors of NK-like and allospecific cytotoxic effector cells. J Immunol, 1984. 133(2): p. 702-8.
17. Pattengale, P. K., et al., Lysis of fresh leukemic blasts by interferon-activated human natural killer cells. Nat Immun Cell Growth Regul, 1983. 3(4): p. 165-80.
18. Stewart, L. D. and E. W. Ades, Human natural killer cells: correlation of a lytic assay with a visual binding assay. J Clin Lab Immunol, 1983. 10(4): p. 203-8.
19. Golay, J., et al., Cord blood-derived cytokine-induced killer cells combined with blinatumomab as a therapeutic strategy for CD19(+) tumors. Cytotherapy, 2018. 20(8): p. 1077-1088.
20. Rettman, P., et al., New insights on the natural killer cell repertoire from a thorough analysis of cord blood cells. J Leukoc Biol, 2016. 100(3): p. 471-9.
21. Tomchuck, S. L., W. H. Leung, and M. H. Dallas, Enhanced cytotoxic function of natural killer and CD3+CD56+ cells in cord blood after culture. Biol Blood Marrow Transplant, 2015. 21(1): p. 39-49.
22. Cany, J., et al., Natural killer cells generated from cord blood hematopoietic progenitor cells efficiently target bone marrow-residing human leukemia cells in NOD/SCID/IL2Rg(null) mice. PLoS One, 2013. 8(6): p. e64384.
23. Lin, S. J. and M. L. Kuo, Cytotoxic function of umbilical cord blood natural killer cells: relevance to adoptive immunotherapy. Pediatr Hematol Oncol, 2011. 28(8): p. 640-6.
24. Wei, Y., et al., Ex vivo expansion of CD34+ and T and NK cells from umbilical cord blood for leukemic BALB/C nude mouse transplantation. Int J Hematol, 2008. 87(2): p. 217-224.
25. Stikvoort, A., et al., CD38-specific Chimeric Antigen Receptor Expressing Natural Killer KHYG-1 Cells: A Proof of Concept for an “Off the Shelf” Therapy for Multiple Myeloma. Hemasphere, 2021. 5(7): p. e596.
26. Yu, X. X., et al., Effect of the in vivo application of granulocyte colony-stimulating factor on NK cells in bone marrow and peripheral blood. J Cell Mol Med, 2018. 22(6): p. 3025-3034.
27. van Gelder, M., et al., Bone marrow produces sufficient alloreactive natural killer (NK) cells in vivo to cure mice from subcutaneously and intravascularly injected 4T1 breast cancer. Breast Cancer Res Treat, 2017. 161(3): p. 421-433.
28. Terrazzano, G., et al., Natural killer expansion, human leukocyte antigens-E expression and CD14(+) CD56(+) monocytes in a myelodysplastic syndrome patient. Eur J Haematol, 2013. 91(3): p. 265-9.
29. Taveirne, S., et al., The transcription factor ETS1 is an important regulator of human NK cell development and terminal differentiation. Blood, 2020. 136(3): p. 288-298.
30. Dege, C., et al., Potently Cytotoxic Natural Killer Cells Initially Emerge from Erythro-Myeloid Progenitors during Mammalian Development. Dev Cell, 2020. 53(2): p. 229-239 e7.
31. Bernareggi, D., S. Pouyanfard, and D. S. Kaufman, Development of innate immune cells from human pluripotent stem cells. Exp Hematol, 2019. 71: p. 13-23.
32. Zhu, H. and D. S. Kaufman, An Improved Method to Produce Clinical-Scale Natural Killer Cells from Human Pluripotent Stem Cells. Methods Mol Biol, 2019. 2048: p. 107-119.
33. Fontaine, M. J., et al., Variations in novel cellular therapy products manufacturing. Cytotherapy, 2020. 22(6): p. 337-342.
34. Dang, N., et al., Donor Lymphocyte-Derived Natural Killer Cells Control MHC Class I-Negative Melanoma. Cancer Immunol Res, 2020. 8(6): p. 756-768.
35. Ciurea, S. O., et al., Phase 1 clinical trial using mbIL21 ex vivo-expanded donor-derived NK cells after haploidentical transplantation. Blood, 2017. 130(16): p. 1857-1868.
36. Hosseini, E., et al., Ex vivo expansion of CD3(depleted) cord blood-MNCs in the presence of bone marrow stromal cells; an appropriate strategy to provide functional NK cells applicable for cellular therapy. Stem Cell Res, 2017. 19: p. 148-155.
37. Mahajan, D., et al., Role of Natural Killer Cells during Pregnancy and Related Complications. Biomolecules, 2022. 12(1).
38. Jiang, L., et al., Extracellular Vesicle-Mediated Secretion of HLA-E by Trophoblasts Maintains Pregnancy by Regulating the Metabolism of Decidual NK Cells. Int J Biol Sci, 2021. 17(15): p. 4377-4395.
39. Zhao, H., R. J. Wong, and D. K. Stevenson, The Impact of Hypoxia in Early Pregnancy on Placental Cells. Int J Mol Sci, 2021. 22(18).
40. Yang, X. and T. Meng, Killer-cell immunoglobulin-like receptor/human leukocyte antigen-C combination and ‘great obstetrical syndromes’ (Review). Exp Ther Med, 2021. 22(4): p. 1178.
41. Zhang, J., et al., Ovarian stimulation reduces fetal growth by dysregulating uterine natural killer cells in mice. Mol Reprod Dev, 2021. 88(9): p. 618-627.
42. Wadhwa, L. and A. Rani, Impact of Intrauterine Administration of Human Chorionic Gonadotropin before Intrauterine Insemination in Infertile Women: A Randomized Controlled Trial. J Hum Reprod Sci, 2021. 14(2): p. 156-161.
43. Radomska-Lesniewska, D. M., A. Bialoszewska, and P. Kaminski, Angiogenic Properties of NK Cells in Cancer and Other Angiogenesis-Dependent Diseases. Cells, 2021. 10(7).
44. Hebert, J. F., et al., Male fetal sex affects uteroplacental angiogenesis in growth restriction mouse modeldagger. Biol Reprod, 2021. 104(4): p. 924-934.
45. Hadas, R., et al., Hyaluronan control of the primary vascular barrier during early mouse pregnancy is mediated by uterine NK cells. JCI Insight, 2020. 5(22).
46. Jafarpour, R., et al., Functional significance of lymphocytes in pregnancy and lymphocyte immunotherapy in infertility: A comprehensive review and update. Int Immunopharmacol, 2020. 87: p. 106776.
47. Zheng, J., et al., Pigment epithelium-derived factor, a novel decidual natural killer cells-derived factor, protects decidual stromal cells via anti-inflammation and anti-apoptosis in early pregnancy. Hum Reprod, 2020. 35(7): p. 1537-1552.
48. Dickson, M. A., et al., Carbon monoxide increases utero-placental angiogenesis without impacting pregnancy specific adaptations in mice. Reprod Biol Endocrinol, 2020. 18(1): p. 49.
49. Phillips, J. H., et al., Ontogeny of human natural killer (NK) cells: fetal NK cells mediate cytolytic function and express cytoplasmic CD3 epsilon, delta proteins. J Exp Med, 1992. 175(4): p. 1055-66.
50. Manoussaka, M., et al., Phenotypic and functional characterization of long-lived NK cell lines of different maturational status obtained from mouse fetal liver. J Immunol, 1997. 158(1): p. 112-9.
51. Lauzurica, P., et al., Phenotypic and functional characteristics of hematopoietic cell lineages in CD69-deficient mice. Blood, 2000. 95(7): p. 2312-20.
52. Takei, F., et al., Ly49 and CD94/NKG2: developmentally regulated expression and evolution. Immunol Rev, 2001. 181: p. 90-103.
53. Brooks, C. G., A. Georgiou, and R. K. Jordan, The majority of immature fetal thymocytes can be induced to proliferate to IL-2 and differentiate into cells indistinguishable from mature natural killer cells. J Immunol, 1993. 151(12): p. 6645-56.
54. Carlyle, J. R., et al., Natural killer cell development and function precede alpha beta T cell differentiation in mouse fetal thymic ontogeny. J Immunol, 1998. 160(2): p. 744-53.
55. Durum, S. K., et al., CD16 cross-linking blocks rearrangement of the TCR beta locus and development of alpha beta T cells and induces development of NK cells from thymic progenitors. J Immunol, 1998. 161(7): p. 3325-9.
56. Lee, C. K., et al., Cloning thymic precursor cells: demonstration that individual pro-Tl cells have dual T-NK potential and individual pro-T2 cells have dual alphabeta-gammadelta T cell potential. Cell Immunol, 1999. 191(2): p. 139-44.
57. Melder, R. J., et al., Modulation of A-NK cell rigidity: In vitro characterization and in vivo implications for cell delivery. Biorheology, 2001. 38(2-3): p. 151-9.
58. Yoshida, H., et al., Expression of alpha(4)beta(7) integrin defines a distinct pathway of lymphoid progenitors committed to T cells, fetal intestinal lymphotoxin producer, NK, and dendritic cells. J Immunol, 2001. 167(5): p. 2511-21.
59. DiSanto, J. P., et al., Lymphoid development in mice with a targeted deletion of the interleukin 2 receptor gamma chain. Proc Natl Acad Sci USA, 1995. 92(2): p. 377-81.
60. Yokota, Y., et al., Development of peripheral lymphoid organs and natural killer cells depends on the helix-loop-helix inhibitor Id2. Nature, 1999. 397(6721): p. 702-6.
61. Saito, Y., et al., The in vivo development of human T cells from CD34(+) cells in the murine thymic environment. Int Immunol, 2002. 14(10): p. 1113-24.
62. Martin, C. H., et al., Differences in lymphocyte developmental potential between human embryonic stem cell and umbilical cord blood-derived hematopoietic progenitor cells. Blood, 2008. 112(7): p. 2730-7.
63. Samarina, A. M., O. V. Podgornyi, and M. A. Aleksandrova, Analysis of differentiation of stem/progenitor cells in tissue culture of neocortical primordium of mouse brain. Bull Exp Biol Med, 2007. 144(4): p. 602-7.
64. Poggi, A. and M. C. Mingari, Development of human NK cells from the immature cell precursors. Semin Immunol, 1995. 7(2): p. 61-6.
65. Strobl, H., et al., Flow cytometric analysis of intracellular CD68 molecule expression in normal and malignant haemopoiesis. Br J Haematol, 1995. 90(4): p. 774-82.
66. Nakayama, N., I. Fang, and G. Elliott, Natural killer and B-lymphoid potential in CD34+ cells derived from embryonic stem cells differentiated in the presence of vascular endothelial growth factor. Blood, 1998. 91(7): p. 2283-95.
67. Toomey, J. A., et al., Stochastic acquisition of Qal receptors during the development of fetal NK cells in vitro accounts in part but not in whole for the ability of these cells to distinguish between class I-sufficient and class I-deficient targets. J Immunol, 1999. 163(6): p. 3176-84.
68. Bodey, B., et al., Fas (APO-1, CD95) receptor expression and new options for immunotherapy in childhood medulloblastomas. Anticancer Res, 1999. 19(4B): p. 3293-314.
69. Nakayama, N., J. Lee, and L. Chiu, Vascular endothelial growth factor synergistically enhances bone morphogenetic protein-4-dependent lymphohematopoietic cell generation from embryonic stem cells in vitro. Blood, 2000. 95(7): p. 2275-83.
70. Lian, R. H., et al., Orderly and nonstochastic acquisition of CD94/NKG2 receptors by developing NK cells derived from embryonic stem cells in vitro. J Immunol, 2002. 168(10): p. 4980-7.
71. Prost, S., et al., Characterization of a novel hematopoietic marker expressed from early embryonic hematopoietic stem cells to adult mature lineages. Blood Cells Mol Dis, 2002. 29(2): p. 236-48.
72. Woll, P. S., et al., Human embryonic stem cell-derived NK cells acquire functional receptors and cytolytic activity. J Immunol, 2005. 175(8): p. 5095-103.
73. Tabatabaei-Zavareh, N., et al., Characterization of developmental pathway of natural killer cells from embryonic stem cells in vitro. PLoS One, 2007. 2(2): p. e232.
74. McCullar, V., et al., Mouse fetal and embryonic liver cells differentiate human umbilical cord blood progenitors into CD56-negative natural killer cell precursors in the absence of interleukin-15. Exp Hematol, 2008. 36(5): p. 598-608.
75. Jiang, Q., et al., Modifying strategies for SDF-1/CXCR4 interaction during mesenchymal stem cell transplantation. Gen Thorac Cardiovasc Surg, 2022. 70(1): p. 1-10.
76. Oprescu, N., et al., Inflammatory markers in acute myocardial infarction and the correlation with the severity of coronary heart disease. Ann Med, 2021. 53(1): p. 1041-1047.
77. Esmaeili, R., et al., Mesenchymal Stem Cells Pretreatment With Stromal-Derived Factor-1 Alpha Augments Cardiac Function and Angiogenesis in Infarcted Myocardium. Am J Med Sci, 2021. 361(6): p. 765-775.
78. Zacherl, M. J., et al., Molecular imaging of cardiac CXCR4 expression in a mouse model of acute myocardial infarction using a novel (68)Ga-mCXCL12 PET tracer. J Nucl Cardiol, 2021. 28(6): p. 2965-2975.
79. Chen, B. and N. G. Frangogiannis, Chemokines in Myocardial Infarction. J Cardiovasc Transl Res, 2021. 14(1): p. 35-52.
80. Schaun, M. I., et al., Physical training prior to myocardial infarction potentializes stem cell therapy, SDF-1/CXCR4 axis activation and inhibits the vasoconstrictor response in hypertensive rats. Cytokine, 2020. 126: p. 154912.
81. Song, Y. L., et al., Mesenchymal Stem Cell-Platelet Aggregates Increased in the Peripheral Blood of Patients with Acute Myocardial Infarction and Might Depend on the Stromal Cell-Derived Factor 1/CXCR4 Axis. Stem Cells Dev, 2019. 28(24): p. 1607-1619.
82. Spiller, S., et al., Modulation of Human CXCL12 Binding Properties to Glycosaminoglycans To Enhance Chemotactic Gradients. ACS Biomater Sci Eng, 2019. 5(10): p. 5128-5138.
83. Huang, P., et al., Combinatorial treatment of acute myocardial infarction using stem cells and their derived exosomes resulted in improved heart performance. Stem Cell Res Ther, 2019. 10(1): p. 300.
84. Peiro, 0.M., et al., Stromal cell derived factor-1 and long-term prognosis in acute coronary syndrome. Biomark Med, 2019. 13(14): p. 1187-1198.
85. Li, M., et al., Vildagliptin and G-CSF Improved Angiogenesis and Survival after Acute Myocardial Infarction. Arch Med Res, 2019. 50(3): p. 133-141.
86. Huang, F. Y., et al., The bifunctional SDF-1-AnxA5 fusion protein protects cardiac function after myocardial infarction. J Cell Mol Med, 2019. 23(11): p. 7673-7684.
87. Gong, X. H., et al., Exosomes derived from SDF1-overexpressing mesenchymal stem cells inhibit ischemic myocardial cell apoptosis and promote cardiac endothelial microvascular regeneration in mice with myocardial infarction. J Cell Physiol, 2019. 234(8): p. 13878-13893.
88. Ciullo, A., et al., Exosomal Expression of CXCR4 Targets Cardioprotective Vesicles to Myocardial Infarction and Improves Outcome after Systemic Administration. Int J Mol Sci, 2019. 20(3).
89. Yuan, Z., et al., 17beta-estradiol promotes recovery after myocardial infarction by enhancing homing and angiogenic capacity of bone marrow-derived endothelial progenitor cells through ERalpha-SDF-1/CXCR4 crosstalking. Acta Biochim Biophys Sin (Shanghai), 2018. 50(12): p. 1247-1256.
90. Goldstone, A. B., et al., SDF 1-alpha Attenuates Myocardial Injury Without Altering the Direct Contribution of Circulating Cells. J Cardiovasc Transl Res, 2018. 11(4): p. 274-284.
91. Renko, 0., et al., SDF1 gradient associates with the distribution of c-Kit+cardiac cells in the heart. Sci Rep, 2018. 8(1): p. 1160.
92. Huynh, C., et al., Relevance of the CXCR4/CXCR7-CXCL12 axis and its effect in pathophysiological conditions. Pharmacol Res, 2020. 161: p. 105092.
93. Song, A., et al., The Role of CXCL12 in Kidney Diseases: A Friend or Foe? Kidney Dis (Basel), 2021. 7(3): p. 176-185.
94. Chang, H. M., et al., FGF23 ameliorates ischemia-reperfusion induced acute kidney injury via modulation of endothelial progenitor cells: targeting SDF-1/CXCR4 signaling. Cell Death Dis, 2021. 12(5): p. 409.
95. Bennington, J., et al., Chemokine Therapy in Cats With Experimental Renal Fibrosis and in a Kidney Disease Pilot Study. Front Vet Sci, 2021. 8: p. 646087.
96. Sun, Z., et al., Stromal cell-derived factor-1/CXC chemokine receptor 4 axis in injury repair and renal transplantation. J Int Med Res, 2019. 47(11): p. 5426-5440.
97. Gridelet, V., et al., Human Chorionic Gonadotrophin: New Pleiotropic Functions for an “Old” Hormone During Pregnancy. Front Immunol, 2020. 11: p. 343.
98. Liu, J., et al., Low-energy shock wave pretreatment recruit circulating endothelial progenitor cells to attenuate renal ischaemia reperfusion injury. J Cell Mol Med, 2020. 24(18): p. 10589-10603.

1. A method of treating ovarian degeneration comprising administration of natural killer (NK) cells which have been modified to allow for enhanced regenerative activity wherein said modification comprising contacting said NK cells with a regenerative cell population.

2. The method of claim 1, wherein said natural killer cell are either autologous, allogeneic, or xenogeneic to said patient.

3. The method of claim 2, wherein said natural killer cell line is NK-92 or a derivative thereof.

4. The method of claim 1, wherein said natural killer cell is derived from a pluripotent stem cell.

5. The method of claim 4, wherein said pluripotent stem cell is an inducible pluripotent stem cell.

6. The method of claim 1, wherein said enhanced survival and/or activity is accomplished through treatment of said natural killer cells with hypoxic preconditioning.

7. The method of claim 6, wherein said hypoxic preconditioning comprises exposure of said natural killer cells to conditions allowing for upregulation of HIF-1 alpha gene expression by 25% or more as compared to baseline culture.

8. The method of claim 1, wherein modification of said NK cell to allow for enhanced survival and/or activity constitutes culturing said NK cell with an epigenetic modulator.

9. The method of claim 8, wherein said epigenetic modulator comprises a histone deacetylase inhibitor.

10. The method of claim 9, wherein said histone deacetylase inhibitor is trichostatin A.

11. The method of claim 9, wherein said histone deacetylase inhibitor is mocetinostat.

12. The method of claim 1, wherein T cells are generated to possess a hypoxia resistant phenotype instead of NK cells.

13. The method of claim 1, wherein said NK cells are cultured with regenerative cells.

14. The method of claim 13, wherein said culture comprises addition of interleukin-12.

15. The method of claim 13, wherein said culture comprises addition of interleukin-15.

16. The method of claim 13, wherein said culture comprises addition of culture supernatant from said regenerative cells to said NK cells.

17. The method of claim 13, wherein said regenerative cells are activated by culture with an inflammatory cytokine.

18. The method of claim 13, wherein said regenerative cells are activated by culture with an activator of NF-kappa B.

19. The method of claim 13, wherein said regenerative cells are activated by treatment with interferon gamma.

20. The method of claim 13, wherein said regenerative cells are activated by treatment with neutrophil extracellular traps.
